(19) United States
(12) Patent Application Publication
(10) Pub. No.: US 2024/0418679 A1
(43) Pub. Date: Dec. 19, 2024

(54) DEVICE FOR PERFORMING NON-DESTRUCTIVE CONTROL OF A RAIL

(71) Applicant: PCM SRL SOCIO UNICO, RAVENNA (IT)

(72) Inventor: Marco Salciccia, RAVENNA (IT)

(73) Assignee: PCM SRL SOCIO UNICO, RAVENNA (IT)

(21) Appl. No.: 18/701,823

(22) PCT Filed: Nov. 14, 2022
(86) PCT No.: PCT/IB2022/060926

§ 371 Date: Apr. 16, 2024

(30) Foreign Application Priority Data
Nov. 16, 2021 (IT) 102021000029039

(51) Int. Cl. G01N 29/06; B61K 9/08; G01N 29/265; G01N 33/207

(52) U.S. Cl. CPC G01N 29/06; B61K 9/08; G01N 29/265; G01N 33/207; G01N 2291/2623

(57) ABSTRACT

A device for performing non-destructive control of a rail that develops along an axis, the device comprising a support frame designed to be positioned in the vicinity of the rail, and an ultrasound detection unit movable relative to the frame, the device further comprising an anthropomorphic robot having a base connected to the frame and an end portion connected to the detection unit; said robot is designed to move the detection unit relative to the rail in a first direction substantially parallel in use to the axis, in a second direction substantially perpendicular to the first direction and in a third direction substantially perpendicular to the first direction and second direction.

The present invention concerns a device for the non-destructive control of a rail. In particular, the present invention refers to a device comprising a frame, an ultrasound detection unit and an anthropomorphic robot designed to move the detection unit relative to the frame.

### DESCRIPTION OF THE STATE OF THE ART

In the railway superstructure sector, the need to perform periodic non-destructive controls on the state of the rails that compose the tracks has been felt for some time, in order to promptly identify any defects which in the long term could deteriorate until compromising operation of the track.

Generally, each rail is made by butt-welding a plurality of steel profiles with length comprised between 12 metres and 108 metres, having a substantially double T-shaped section; more specifically, said section has an upper head designed to define the rolling surface for transit of the rolling stock, a lower foot resting on the track sleepers and having transverse width greater than that of the head, and a web (or core) arranged vertically to join the head and the foot. Usually, the profiles are head-joined by aluminothermic welding: this process is particularly delicate, as it is carried out in situ on the profiles already fixed to the sleepers, therefore outdoors and in temperature and humidity conditions which are not easy to control.

Consequently, in the context of controls on the state of the rails, the checks on the welds between the profiles are of crucial importance, since defects are often found in the area of these joints, due to the frequent sub-optimal ambient conditions in which the aluminothermic welding operation is carried out.

In the technical field of reference, different solutions are known for performing non-destructive controls on the welds between the profiles that compose the rails. The most common solution entails the use of an ultrasound probe which is positioned in contact with the surface of the rail, and moved manually by an operator along said surface: the probe is designed to emit high frequency sound waves which propagate inside the rail, and to capture said waves once they have undergone reflection. By moving the probe along the surface of the rail, and comparing the different wave reflection patterns obtained, it is possible to identify the presence of any defects in the weld.

This solution allows rapid control of the welds, at the same time guaranteeing operator safety, but it has the drawback of being unreliable and difficult to reproduce: it has been observed, in fact, that the result of the control depends to a large extent on the ability and experience of the operator who moves the probe, since the variation in the reflection pattern of the ultrasounds inside the rail is significantly influenced by the way in which the probe is moved along the relative surface. Furthermore, this solution can only be used for control of the head and web of the rail, since propagation of the sound waves in the foot produces a much more complicated, and even more dependent on the way in which the probe is moved, reflection pattern.

As an alternative to this procedure, the use of X-ray scanning devices is often proposed to detect the presence of any defects in the welds between the profiles. This solution is much more reliable and reproducible than the use of ultrasound waves, due to the high penetration capacity of the electromagnetic waves used, but poses important safety problems: the operators responsible for the controls have to be provided with adequate radiation screening equipment, while the rest of the personnel have to be moved a sufficient distance away from the area. This requirement makes the weld control procedure particularly slow and costly.

Other fairly common solutions for control of the welds between the profiles consist in the use of penetrating liquids or magnetic detectors: however, both methods only allow the identification of surface defects, or defects located just below the surface of the rail, and are ineffective in identifying in-depth defects.

The problem of creating a device and a procedure that allow rapid, reliable and reproducible control of the welds between the profiles composing the rails, at the same time guaranteeing the safety of the operators involved, is currently unsolved, and represents an interesting challenge for the Applicant.

In the light of the situation described above, it would be desirable to have an inexpensive and practical-to-use device that can limit, and if possible overcome, the drawbacks typical of the state of the art.

### SUMMARY OF THE PRESENT INVENTION

The present invention concerns a device for the non-destructive control of a rail. In particular, the present invention refers to a device comprising a frame, an ultrasound detection unit and an anthropomorphic robot designed to move the detection unit relative to the frame. The drawbacks described above are solved by the present invention according to at least one of the following claims.

According to some embodiments of the present invention, a device is provided to perform the non-destructive control of a rail that develops along an axis, said device comprising a support frame designed to be positioned in the vicinity of said rail, and an ultrasound detection unit movable relative to said frame, characterised in that it comprises an anthropomorphic robot having a base connected to said frame and an end portion connected to said detection unit, said robot being designed to move said detection unit relative to said rail in a first direction substantially parallel in use to said axis, in a second direction substantially perpendicular to said first direction and in a third direction substantially perpendicular to said first direction and second direction.

According to an embodiment as described above, said detection unit comprises a support carried in a respective median point by said end portion, said support being rotatable relative to said end portion around a central rotation axis substantially perpendicular to said first direction and passing through said median point, a first ultrasound probe and a second ultrasound probe being carried by said support on opposite sides with respect to said median point.

According to an embodiment as described above, said detection unit comprises a first post and a second post carried by said support on opposite sides with respect to to said median point, said first post and second post being rotatable relative to said support respectively around a first rotation axis and a second rotation axis substantially parallel to said central rotation axis, said first probe and second probe being carried respectively by said first post and second post.

According to an embodiment as described above, at least one of said first post and second post can be translated along said support.

According to an embodiment as described above, said first probe and second probe are of the phased array type.

According to an embodiment as described above, said detection unit comprises a third ultrasound probe of the conventional type with a single element carried by said first post and having a third emission direction substantially perpendicular to said first direction, and/or a fourth ultrasound probe of the conventional type with a double element carried by said second post and having a fourth emission direction substantially perpendicular to said first direction.

According to an embodiment as described above, said robot is designed to incline said detection unit around an inclination axis substantially parallel to said first direction.

According to an embodiment as described above, said frame comprises coupling means that can be reversibly fixed in use to said rail.

According to an embodiment as described above, said detection unit comprises identification means designed to identify a given position along said rail in which to perform said control.

According to an embodiment as described above, said identification means comprise a laser profilometer designed to identify a central region of a weld of said rail.

### BRIEF DESCRIPTION OF THE FIGURES

Further characteristics and advantages of the device according to the present invention will appear clearer from the following description, provided with reference to the attached figures which illustrate at least one non-limiting embodiment example. In particular:

FIG. 1 is a three-dimensional perspective view of a preferred non-limiting embodiment of a device according to the present invention;

FIG. 2 is an enlarged frontal view of FIG. 1;

FIG. 3 is an enlarged three-dimensional perspective view of a detection unit taken from FIG. 1;

FIG. 4 is a lateral view of FIG. 3.

### DETAILED DISCLOSURE OF THE PRESENT INVENTION

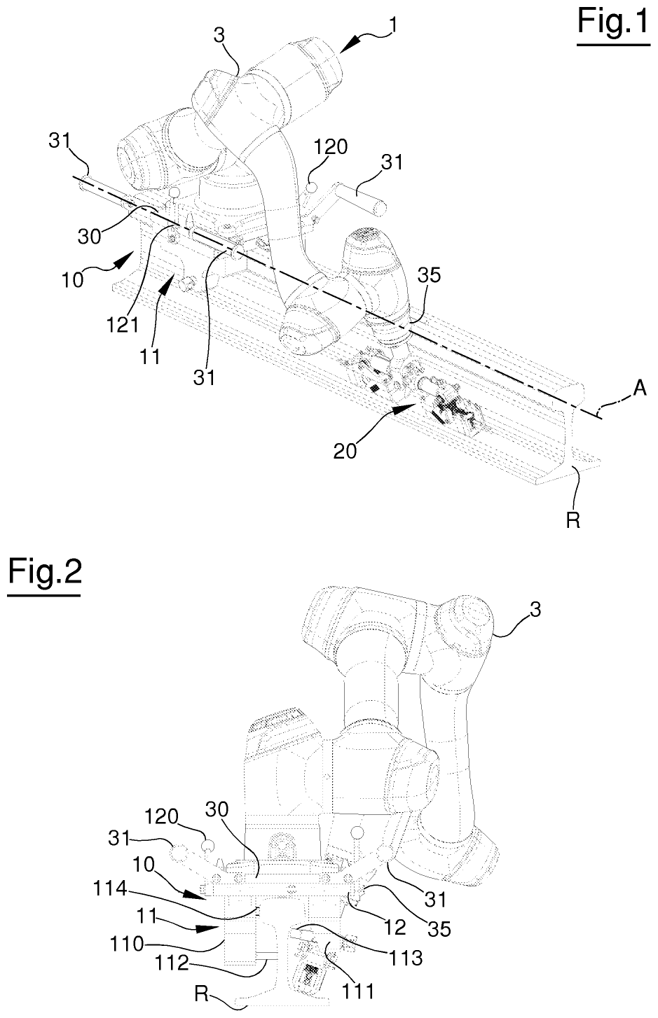

In FIG. 1, the number 1 indicates overall a device for performing the non-destructive control of a rail R that develops along an axis A. In particular, if the rail R is rectilinear, the axis A shall be identified by the direction in which the rail R is arranged, whereas if the rail R is curvilinear, the axis A shall be identified by the direction tangent to the rail R at the point in which the control is performed.

The device 1 firstly comprises a support frame 10 designed to be positioned in the vicinity of the rail R. Preferably the frame 10 comprises in turn coupling means 11 which can be reversibly fixed to the rail R, so that the entire frame 10 can be reversibly fixed to the rail R in a position that can be defined as required, thus being supported by the rail R.

With reference to FIG. 2, the coupling means 11 comprise a first plate 110 and a second plate 111 extending parallel to the axis A on the opposite sides of the rail R, and a plurality of keys 112, 113, 114 which engage respective holes provided on the first plate 110 and on the second plate 111 and which are designed to make contact with the surface of the rail R to lock the frame 10 on the rail R. The frame 10 further comprises a third plate 12 arranged transversally to the axis A which connects the first plate 110 and the second plate 111, resting on the upper surface of the head of the rail R.

Again with reference to FIG. 1, the device 1 further comprises an anthropomorphic robot 3 having a base 30 that can be reversibly fixed to the frame 10. In particular, the base 30 can be positioned on the third plate 12 and has two pins that can be coupled respectively to two locking levers 120, 121 pivoting on the third plate 12: in this way it is possible to provide a rapid, robust and reversible connection between the frame 10 and the base 30 of the robot 3. The base 30 furthermore has a plurality of handles 31 designed to allow manual movement of the robot 3, and the positioning thereof on the frame 10.

The robot 3 further comprises a plurality of arms reciprocally articulated by means of revolving joints to form a chain, said arms extending from the base 30 to an end portion 35, and a plurality of electric actuators designed to rotate the various arms relative to one another. FIG. 2 shows a robot 3 having six movable arms and six respective revolving joints: in this way it is possible to move the end portion 35 (coinciding with the last articulated arm of the chain) relative to the base 30 in three linear directions, and orient it according to two angles.

The device 1 further comprises an ultrasound detection unit 20 carried by the end portion 35 of the robot 3: in this way the robot 3 is able to move the detection unit 20 relative to the frame 10, and therefore relative to the rail R, in a first direction X substantially parallel to the axis A, in a second direction Y substantially perpendicular to the first direction X, and in a third direction Z substantially perpendicular to the first direction X and to the second direction Y. Furthermore, the robot 3 is able to orient the detection unit 20 according to two angles, and therefore in particular incline it around a first inclination axis substantially parallel to the first direction X.

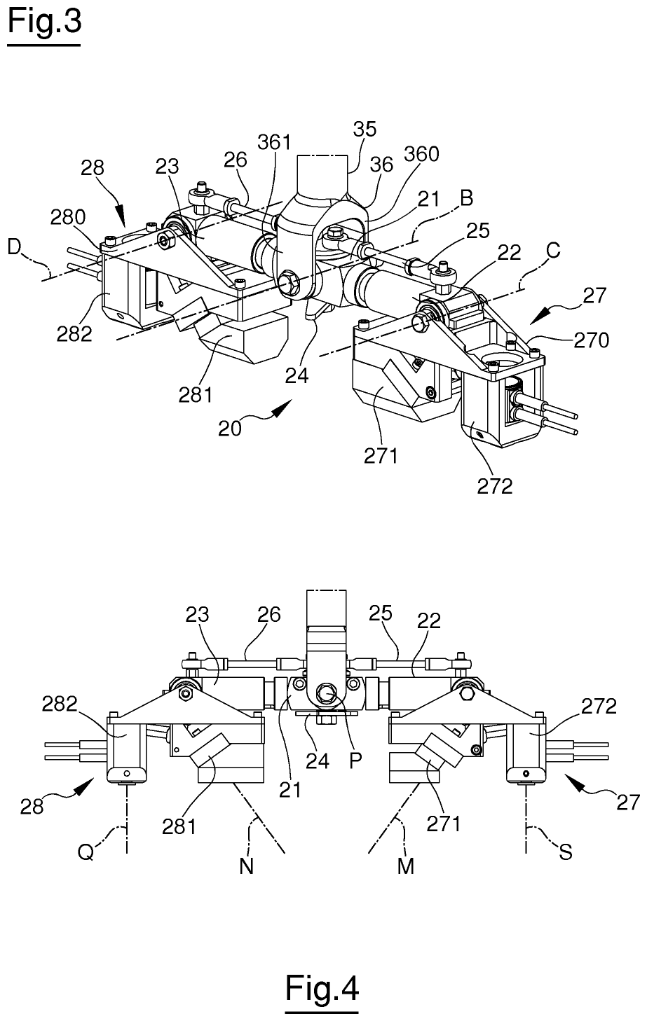

With reference to FIG. 3, the end portion 35 of the robot 3 terminates in a fork 36 having a first arm 360 and a second arm 361 which extend parallel to each other, defining a seat in the middle thereof. The detection unit 20 comprises a support 21 hinged in a respective median point P (shown in FIG. 4) to the first arm 360 and to the second arm 361 so as to occupy the seat and rotate relative to the end portion 35 around a central rotation axis B passing through the median point P, which in use is substantially perpendicular to the axis A of the rail R.

The detection unit 20 further comprises a first carriage 22 and a second carriage 23 carried by the support 21 on opposite sides with respect to the median point P, said carriages 22, 23 being slidable along the support 21 away from and nearer to the median point P. More specifically, the support 21 has two cylindrical guides that extend on opposite sides with respect to the median point P, and the carriages 22, 23 have respective attachment portions designed to engage the two guides. The translatory movement of the carriages 22, 23 can be manually controlled by acting on a crank 24, which is fixed on the support 21 so as to be able to rotate relative to it around an axis perpendicular to the central rotation axis B, and is connected to the first carriage 22 and to the second carriage 23 by a first connecting rod 25 and a second connecting rod 26 respectively.

The first carriage 22 carries a first probe assembly 27, which comprises a first post 270 hinged to the first carriage 22 so as to be able to rotate relative to it around a first rotation axis C. The first probe assembly 27 further comprises a first ultrasound probe 271 and a third ultrasound probe 272 fixed to the first post 270.

Analogously, the second carriage 23 carries a second probe assembly 28, which comprises a second post 280 hinged to the second carriage 23 so as to be able to rotate relative to it around a second rotation axis D. The second probe assembly 27 further comprises a second ultrasound probe 281 and a fourth ultrasound probe 282 fixed to the second post 280.

With reference to FIG. 4, the first probe 271 and the second probe 281 are of the phased array type. This expression indicates that the first probe 271 and the second probe 282 have respectively a first array and a second array of ultrasound wave emission sources; said source arrays are controlled so as to generate overall parallel wave fronts that are propagated respectively in a first emission direction M and a second emission direction N which can be defined as required by simply varying the sequence in which the emission sources are activated. The first emission direction M and the second emission direction N are oriented so as to be intersecting with each other.

The third probe 272 is of the conventional type with a single element. This expression indicates that the third probe 272 has one single ultrasound wave emission source, which also acts as a detection element, and therefore also has a third well-defined emission direction S. The third probe 253 is oriented so that the third emission direction S is arranged perpendicular to the first direction X, namely, in use, perpendicular to the axis A of the rail R. The fourth probe 282 is of the conventional type with double element. This expression indicates that the fourth probe 282 has one single ultrasound wave emission source, and a detection element alongside said source.

Consequently, also in this case a fourth emission direction Q is well defined, and in addition a depth is also defined at which the scan reaches maximum precision. The fourth probe 282 is oriented so that the fourth emission direction Q is arranged perpendicular to the first direction X, namely, in use, perpendicular to the axis A of the rail R. The device 1 further comprises identification means (not shown in the figure) carried by the support 21 and designed to identify a given position in which to perform the control along the rail R. In particular the identification means comprise a laser profilometer, adapted to identify the central region of a weld between two profiles that compose the rail R, in order to allow the most suitable positioning of the detection unit 20 relative to the rail R by means of the robot 3.

The device 1 lastly comprises electronic control means, known and not illustrated, designed to control operation of the robot 3, so as to control the positioning of the detection unit 20 as required. Furthermore, the electronic control means are designed to command and control operation of the probes 271, 272, 281, 282, to perform the control on the rail R. The electronic control means are lastly designed to receive data from the probes 271, 272, 281, 282, and to send said data to a remote operations centre: at this remote operations centre, the data can be collected, analysed and presented to the personnel in charge, so as to allow the geographical localization of any defects present in the rail R and provide information to the personnel concerning the operations necessary for maintenance of the railway line.

In use, the device 1 is used to perform a non-destructive control on a weld of the rail R.

Firstly, the frame 10 is positioned on the rail R in the vicinity of the weld to be controlled, and is fixed to the rail R by means of the coupling means 11. The robot 3 is then fixed to the frame 10, placing the relative base 30 on the third plate 12 of the frame 10 and coupling the pins of the base 30 with the locking levers 120, 121.

The robot 3 is then operated to move the detection unit 20 in the first direction X, the second direction Y and the third direction Z, and to incline it around the first inclination axis, so that the probes 271, 272, 281, 282 are moved on the surface of the rail R to perform the control. In particular, firstly, the central region of the weld is identified by means of the laser profilometer carried by the detection unit 20. The detection unit 20 is then positioned so as to bring the first probe 271 and the second probe 281 into contact with an upper surface of the head of the rail R, so that the central region of the weld is in an intermediate position between the first probe 271 and the second probe 281.

The detection unit 20 is then moved in the second direction Y back and forward to move the first probe 271 and the second probe 281 on the upper surface of the head of the rail R. In this way, a first part of the control of the weld is performed, and in particular of the head, web and central part of the foot of the rail R.

If the welding is completed between profiles positioned at slightly different heights, and therefore there is a “step” between said profiles, the fact that the first probe 271 and the second probe 281 are carried by the support 21, which is rotatable relative to the end portion 35 of the robot 3, allows the first probe 271 and the second probe 281 to be positioned respectively in contact with the upper surfaces of the two profiles, even if they are at slightly different heights. The fact that the first probe 271 and the second probe 281 are fixed respectively to a first post 270 and to a second post 280 rotatable relative to the support 21, furthermore, allows the first probe 271 and the second probe 281 to be always maintained in the correct direction relative to the surface of the rail R.

The detection unit 20 is then positioned so as to bring the third probe 272 into contact with the upper surface of the head of the rail R, so that the central region of the weld is vertically below the third probe 272.

The detection unit 20 is then moved to move the third probe 272 in the first direction X back and forward on the upper surface of the head of the rail R. In this way, a second part of the control is performed on the weld, and in particular on the head, web and central part of the foot of the rail R, in order to detect any defects not found during the first part of the control.

The detection unit 20 is then positioned so as to bring the fourth probe 282 into contact with the upper surface of the head of the rail R, so that the central region of the weld is vertically below the fourth probe 282.

The detection unit 20 is then moved to move the fourth probe 282 in the first direction X back and forward and in the second direction Y back and forward on the upper surface of the head of the rail R. In this way, a third part of the control is performed on the weld, and in particular on the upper portion of the head of the rail R, in order to detect any defects not found during the first part and the second part of the control.

The detection unit 20 is then positioned so as to bring the first probe 271 and the second probe 281 into contact with an upper surface of the foot of the rail R, first on one side of the web and then on the opposite side, so that the central region of the weld is in an intermediate position between the first probe 271 and the second probe 281. In this way, a fourth part of the control of the welding is performed, and in particular of the lateral portions of the foot of the rail R.

In the light of the above description, it is easy to see that the device 1 is perfectly able to solve the drawbacks of the state of the art illustrated above.

In fact, the device 1 allows for automated, reliable and reproducible control of the entire section of a weld of the rail R. Obviously it is also possible to perform only one of the parts of the control, or only some of them in the preferred order, if control of only some portions of the weld section is required.

Furthermore, the same device 1 can also be used to perform a non-destructive control of a hole of a rail joint. In this case the detection unit 20 is positioned so as to bring the first probe 271 and the second probe 281 into contact with an upper surface of the head of the rail R, so that the hole is in an intermediate position between the first probe 271 and the second probe 281. The detection unit 20 is then moved to move the first probe 271 and the second probe 281 in the first direction X back and forward on the upper surface of the head of the rail R, performing the control on the hole.

Lastly, it is clear that modifications and variations can be made to the device described here without departing from the protective scope of the present invention.

1. A device (1) for performing non-destructive control of a rail (R) that develops along an axis (A), said device (1) comprising a support frame (10) designed to be positioned in the vicinity of said rail (R), and an ultrasound detection unit (20) movable relative to said frame (10), characterised in that it comprises an anthropomorphic robot (3) having a base (30) connected to said frame (10) and an end portion (35) connected to said detection unit (20), said robot (3) being designed to move said detection unit (20) relative to said rail (R) in a first direction (X) substantially parallel in use to said axis (A), in a second direction (Y) substantially perpendicular to said first direction (X) and in a third direction (Z) substantially perpendicular to said first direction (X) and second direction (Y).

2. The device (1) according to claim 1, characterised in that said detection unit (20) comprises a support (21) carried in a respective median point (P) by said end portion (35), said support (21) being rotatable relative to said end portion (35) around a central rotation axis (B) substantially perpendicular to said first direction (X) and passing through said median point (P), a first ultrasound probe (271) and a second ultrasound probe (281) being carried by said support (21) on opposite sides with respect to said median point (P).

3. The device (1) according to claim 2, characterised in that said detection unit (20) comprises a first post (270) and a second post (280) carried by said support (21) on opposite sides with respect to said median point (P), said first post (270) and second post (280) being rotatable relative to said support (21) respectively around a first rotation axis (C) and a second rotation axis (D) substantially parallel to said central rotation axis (B), said first probe (271) and second probe (281) being carried respectively by said first post (270) and second post (280).

4. The device (1) according to claim 3, characterised in that at least one of said first post (270) and second post (280) can be translated along said support (21).

5. The device (1) according to claim 3, characterised in that said first probe (271) and second probe (281) are of the phased array type.

6. The device (1) according to claim 5, characterised in that said detection unit (20) comprises a third ultrasound probe (272) of conventional type with single element carried by said first post (270) and having a third emission direction(S) substantially perpendicular to said first direction (X), and/or a fourth ultrasound probe (282) of conventional type with double element carried by said second post (280) and having a fourth emission direction (Q) substantially perpendicular to said first direction (X).

7. The device (1) according to claim 1, characterised in that said robot (3) is designed to incline said detection unit (20) around an inclination axis substantially parallel to said first direction (X).

8. The device (1) according to claim 1, characterised in that said frame (10) comprises coupling means (11) that can be reversibly fixed in use to said rail (R).

9. The device (1) according to claim 1, characterised in that said detection unit (20) comprises identification means designed to identify a given position along said rail (R) in which to perform said control.

10. The device (1) according to claim 9, characterised in that said identification means comprise a laser profilometer designed to identify a central region of a weld of said rail (R).
